SEQUENCE LISTING (5 sequences)

SEQ ID NO: 1 - protein, 33 aa, Artificial
  HIV nef lipopeptide
VGFPVTPQVPLRPMTYKAAVDLSHFLKEKGGLK

SEQ ID NO: 2 - protein, 31 aa, Artificial
  HIV nef lipopeptide
HTQGYFPDWQNYTPGPGVRYPLTFGWLYKLK

SEQ ID NO: 3 - protein, 20 aa, Artificial
  HIV gag lipopeptide
EKIRLRPGGKKKYKLKVIHK

SEQ ID NO: 4 - protein, 33 aa, Artificial
  HIV gag lipopeptide
NPPIPVGEIYKRWIILGLNKIVRMYSPTSILDK

SEQ ID NO: 5 - protein, 32 aa, Artificial
  HIV pol lipopeptide
AIFQSSMTKILEPFRKQNPDIVIYQYMDDLYK

(19) United States
(12) Patent Application Publication
(10) Pub. No.: US 2004/0058861 A1
(43) Pub. Date: Mar. 25, 2004

(54) Use of lipopeptides in immunotherapy of HIV+ individuals

(76) Inventors: Pierre Caudrelier, Lyon (FR); Raphaelle El Habib, Chaponost (FR); Michel Klein, La Tour de Salvagny (FR)

Correspondence Address: Michael S. Greenfield, McDonnell Boehnen Hulbert & Berghoff, 32nd Floor, 300 S. Wacker Drive, Chicago, IL, 60606, US

(21) Appl. No.: 09/948,965

(22) Filed: Sep. 7, 2001

Related U.S. Application Data
(60) Provisional application No. 60/235,079, filed on Sep. 25, 2000.
(60) Provisional application No. 60/278,942, filed on Mar. 27, 2001.

(30) Foreign Application Priority Data
Sep. 8, 2000 (FR) FR0011443
Mar. 2, 2001 (FR) FR0102846

(51) Int. Cl. A61K 38/16

(52) U.S. Cl. 514/12

(57) ABSTRACT

The present invention provides a method for controlling viral rebound in HIV+ individuals after termination of anti-retroviral therapy, the method comprising administering to an HIV+ individual who has undergone anti-retroviral therapy that has been terminated and which HIV+ individual has a viral load of less than or equal to 10,000 copies per ml of plasma and a CD4+ level greater than or equal to 300 cells per mm³, at least one lipopeptide comprising a peptide chain of 7 to 100 amino acids which comprises at least one CTL epitope of an HIV protein and which is linked by covalent attachment, optionally via a linker moiety, to a lipid chain comprising from 8 to 20 carbon atoms.

The present invention discloses a method for treating individuals infected with HIV and relates more particularly to the use of lipopeptides in immunotherapy in HIV+ individuals receiving antiretroviral therapy.

These studies were cofinanced by the ANRS [French association for aids research].

The object of the vast majority of studies carried out on AIDS is to develop a prophylactic method directed toward protecting individuals against infection with HIV. Many antigens have been proposed for this purpose. By way of example, mention may be made of: the HIV envelope glycoprotein, pox viruses expressing epitopes derived from HIV structural proteins or HIV regulatory proteins, and lipopeptides.

The use of lipopeptides has been proposed as an anti-AIDS strategy in particular in FR 90/15870. That document discloses lipopeptides comprising a peptide component having between approximately 10 and 40 amino acids and comprising at least one antigenic determinant, said lipopeptide also comprising one or more chains derived from fatty acids comprising from 10 to 20 carbon atoms, and/or one or more modified steroid groups coupled to αNH₂ or εNH₂ functions of said amino acids. On the basis of an experiment carried out in mice, the authors indicate that said lipopeptides can be used to induce CTLs against any antigenic determinant from any pathogenic agent, including HIV.

The manufacture of mixed micelles or microaggregates containing at least two lipopeptides, one comprising a CTL epitope, the second comprising a T-helper epitope, and their use for inducing an immune response is disclosed in WO 99/27954. According to that document, this formulation makes it possible to induce a response of better quality by adding a helper-T response.

Neither of those two documents either discloses or suggests that the lipopeptides according to the invention can be used to at least temporarily control the viremia in HIV+ individuals receiving antiretroviral therapy, after the antiretroviral treatment has been stopped, as described below.

Several methods for treating HIV have been proposed to date. The only method for treating HIV-related infections which is used at the current time corresponds to a method based on the administration of a combination of antiretroviral medicinal products, known as antiretroviral multitherapy. Although it represents a significant advance in the treatment of AIDS, antiretroviral therapy is far from being an ideal solution. Besides the side effects which are considerable, this type of therapy requires a degree of participation by the patient which is often difficult to obtain. Nonadherence to the treatment results in failure of the treatment and may facilitate the emergence of a virus resistant to the antiviral products.

Various therapeutic strategies have therefore been developed, comprising intermittent interruptions in the antiretroviral therapy and also the use of immunomodulators of the IL2 type.

None of the strategies proposed to date have provided a satisfactory solution. There is therefore a need to set up a method for treating HIV+ individuals which does not have the drawbacks of the methods proposed up until now. Furthermore, any therapeutic strategy which would make it possible to limit the duration of the antiretroviral therapy is highly desirable.

The applicant has demonstrated, surprisingly, that, after infection with HIV, it is possible to stop the antiretroviral therapy if lipopeptides as defined above which induce CD4+ and CD8+ cellular responses specific for HIV are administered. These cellular responses maintain the viral load at low values and control the viral rebound after the antiretroviral therapy has been stopped.

A subject of the present invention is therefore the use of lipopeptides for preparing a vaccine for controlling viral rebound after antiretroviral therapy has been stopped in HIV+ individuals having a viral load of less than or equal to 10,000 copies per ml of plasma and a CD4+ level greater than or equal to 300 cells per mm³, in which the lipopeptides consist of a peptide chain of 7 to 100 amino acids which comprises at least one CTL epitope of an HIV protein, and which is linked by covalent attachment to a lipid chain comprising from 8 to 20 carbon atoms.

According to another embodiment, the HIV+ individuals have a viral load of less than or equal to 50 copies per ml of plasma and a CD4 level of greater than or equal to 500 cells per mm³.

According to one embodiment, the lipopeptides consist of a lipid chain comprising 16 carbon atoms.

According to another embodiment, the lipopeptides correspond to a mixture comprising 5 different lipopeptides having CTL epitopes derived from the Nef, Gag and Pol proteins of HIV.

According to a specific embodiment, the lipopeptide mixture comprises the following lipopeptides:

VGFPVTPQVPLRPMTYKAAVDLSHFLKEKGGLKΣ(Palm) -NH₂
HTQGYFPDWQNYTPGPGVRYPLTFGWLYKLKΣ(Palm) -NH₂
EKIRLRPGGKKKYKLKVIHKΣ(Palm)-NH₂
NPPIPVGEIYKRWIILGLNKIVRMYSPTSILDKΣ(Palm)-NH₂
AIFQSSMTKILEPFRKQNPDIVIYQYMDDLYKΣ(Palm) -NH₂.

According to a specific embodiment, the mixture also comprises a lipopeptide in which the peptide chain consists of a ubiquitous helper epitope.

According to one embodiment, the lipopeptides are administered intramuscularly at a dose of 50 μg to 3 mg of total lipopeptides, preferably in 4 doses on D0, and at 1 month, 2 months and 3 months, respectively.

According to another embodiment, the lipopeptides are co-administered with an attenuated recombinant virus vaccine. Said attenuated recombinant virus is preferably an ALVAC, and in particular an ALVAC vCP1452 or vcp1433.

According to another embodiment, an immunomodulator, preferably IL2, is administered sequentially or simultaneously.

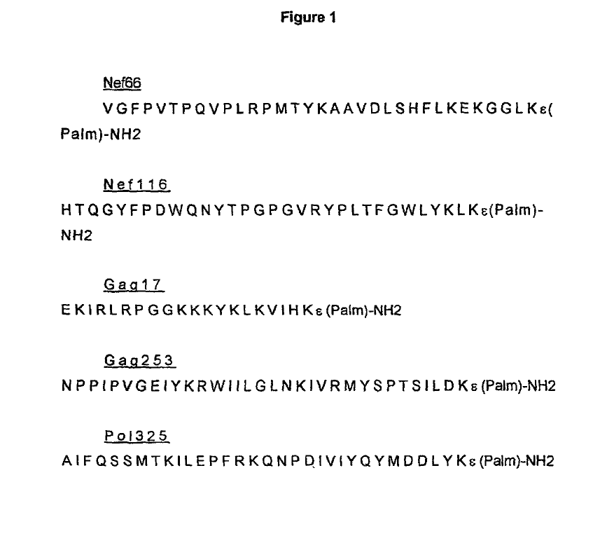

The other characteristics and advantages of the present invention will become apparent in the detailed description which follows with reference to FIG. 1, which gives a schematic representation of the lipopeptides constituting a mixture according to the invention. In FIG. 1, ε(Palm)-NH₂ means that a palmitic acid is linked to the εNH₂ function of the lysine, the COOH-terminal end of the lipid is therefore amidated. The lysine is located at the C-terminal end of the peptide chain. The peptide chain is represented according to the one-letter code in which:

A:Ala; D:Asp; E:GlU; F:Phe:G:Gly; H:His; I:Ile; K:
Lys:L:Leu; M:Met; N:Asn; P:Pro; Q:Gln; R:Arg; S:
Ser; T:Thr; V:Val; W:Trp; and Y:Tyr.

The immunization method according to the present invention can be used for treating individuals who are infected with HIV, who are subjected to antiretroviral therapy and who have a viral load of less than 10,000, preferably less than 5,000, in particular less than 1,000 viral copies/ml of plasma and a CD4+ T-cell level of greater than 300 cells/ml, preferably greater than 500 cells/ml. The patients preferably have a viral load of less than 50 viral copies/ml and a CD4+ level of greater than 500.

The vial load expressed by the number of RNA copies/ml of plasma represents the amount of virus present in the blood. It is also referred to under the terms “viral titer” or “viremia”. Many techniques can be used to measure the viral load of a patient. A review of the state of the art can be found in “Report of the NIH to Define Principles of Therapy of HIV Infection” published in the Morbidity and Mortality Weekly Reports, Apr. 24, 1998, vol. 47, no. RR-5, revised on Jun. 17, 1998, to which reference can be made for a description of the techniques. It is known that HIV replication rates in infected individuals can be measured accurately by measuring the HIV in the plasma. The HIV RNA in the plasma is contained in circular particles of virus or virions, each virion containing 2 copies of the HIV genomic RNA. The concentrations of HIV RNA in the plasma can be quantified either by target amplification (e.g. quantitative polymerase chain reactions [RT-PCR], Amplicor HIV Monitor test, Roche molecular systems) or by nucleic acid sequence-based amplification [NASBA®] (NucliSens™ HIV-1 QT assay, Organon Teknika). The test which was used in the context of the present invention is the Amplicor HIV Monitor test, Roche molecular systems.

The CD4+ T-cell level corresponds to the total number of cells expressing the CD4 marker per mm³ of blood. This level is determined according to the recommendations described in The Morbidity and Mortality Weekly Report, 46(RR-02) Jan. 10, 1997, Centers for Disease Control. In summary, the absolute number of CD4+ in whole blood is measured using a three-phase process. The CD4+ count is the product of three laboratory techniques: the white blood cells (WBC) count; the determination of the percentage of WBCs which are lymphocytes (differential); and the determination of the percentage of lymphocytes which are CD4+ T-cells, by “flow cytometry immunophenotyping”, for example using the FACSCount system from Becton Dickinson.

The term “antiretroviral therapy” is intended to mean a treatment comprising an effective combination of antiretroviral agents. Antiretroviral therapy involves the use of two major categories of medicinal products, i.e. reverse transcriptase inhibitors and protease inhibitors. The reverse transcriptase inhibitors can be nucleoside in nature, such as AZT, ddl, ddC, d4T and 3TC in combination with AZT and Combivir, or non-nucleoside in nature, such as Delavirdine and Nevirapine. A review of the non-nucleoside inhibitors is given in Clinical Care (10197) vol. 9, no. 10, p. 75. Preferably, the antiretroviral therapy corresponds to a highly active therapy (HAART), i.e. a combination of a protease inhibitor, a non-nucleoside reverse transcriptase inhibitor and a nucleoside reverse transcriptase inhibitor, or a combination of two non-nucleoside reverse transcriptase inhibitors and of a nucleoside reverse transcriptase inhibitor.

The patients who may be treated using the method according to the invention are therefore individuals infected with HIV who are subjected to antiretroviral therapy soon after the infection i.e. within the 3 months following the infection, and also the infected individuals who are treated more than 3 months after the infection, the latter being referred to, in the context of the present invention, as chronically infected individuals.

The method according to the invention therefore makes it possible to treat newly infected individuals (i.e. individuals infected for 90 days or less) who are placed on antiretroviral therapy a few months after infection with HIV and therefore exhibit controlled viremia. These individuals have the particularity of giving an incomplete Western Blot. The method according to the invention also makes it possible to treat chronically infected patients receiving antiretroviral therapy. The viremia is controlled when the viral load is maintained at a value of less than 10,000 viral copies per ml of plasma.

HIV+ individuals who exhibit CD4+ and CD8+ cellular responses against the HIV antigens, in particular those who exhibit proliferation of cellular responses against the envelope epitopes (for example gp120) are preferred. Individuals who also exhibit cellular responses against the Gag epitopes (for example p24) are more particularly preferred. HIV+ individuals who have lost their CD4+ and/or CD8+ cellular responses against the HIV antigens may also be immunized using the method according to the invention.

The immunization method according to the present invention makes it possible to control the viral rebound in patients infected with HIV, after the antiretroviral therapy has been stopped.

In the context of the present invention, “control of the viral rebound” means that, after the antiretroviral therapy has been stopped, the viral rebound which appears is delayed or absent, or that the viral load present after the viral rebound (post-rebound set point) is controlled.

The viral rebound is generally observed within the 1 to 3 weeks after the antiretroviral therapy has been stopped. The viral rebound is considered to be delayed when it appears more than a month after the antiretroviral therapy has been stopped. Preferably, the viral rebound appears more than 2 months, and in particular more than 6 months after the antiretroviral therapy has been stopped.

The post-rebound set point represents the plasmatic viral load which is present, in the absence of antiretroviral therapy, after the viral rebound. The post-rebound set point is considered to be controlled when it is maintained at values lower than the values for recommencing the antiretroviral therapy, as defined in the official recommendations published in the various countries, this being for a period of at least 1 month, preferably of at least 2 months, in particular of at least 6 months. These values for recommencing the antiretroviral therapy are typically 10,000 to 50,000 copies of RNA/ml of plasma. Preferably, the post-rebound set point is maintained at values of less than 10,000 viral copies/ml, preferably less than 5000 viral copies/ml, in particular less than 1000 viral copies/ml of plasma.

The present invention therefore provides a method which has the advantage of making it possible to at least temporarily, and preferably definitively, stop the antiretroviral therapy of HIV+ individuals by making it possible to control the phenomenon of viral rebound generally associated with such stopping of the antiretroviral therapy.

Such control of the viral rebound is obtained by administering the lipopeptides according to the invention which induce CD4+ and CD8+ cellular responses specific for HIV.

The term “CD8+ response” is intended to mean the capacity of cytotoxic T cells to recognize and kill cells expressing HIV peptides in the context of class I MHC molecules. Such a response can be measured using various methods well known to those skilled in the art, such as, for example, using the tetramer technique on fresh or cultured PBMCs, or using Elispot assays for gamma INF or functional cytotoxicity assays.

The term “CD4+ response” is intended to mean the capacity of CD4+ T cells to be stimulated or activated by the vaccine according to the invention. The CD4+ responses can be measured using diverse methods well known to those skilled in the art, which have been described above.

In the context of the present invention, the term “lipopeptide” is intended to mean at least one compound consisting of a peptide chain comprising from 7 to 100 amino acids, preferably from 10 to 50 amino acids and more particularly from 20 to 35 amino acids, and of a lipid chain comprising from 8 to 20 carbon atoms, preferably from 14 to 18 carbon atoms, and in particular 16 carbon atoms. The lipopeptides according to the present invention preferably correspond to a mixture of at least two different lipopeptides.

The peptide chain of the lipopeptides according to the present invention comprises at least one CTL epitope of an HIV protein and may also include one or more T-helper epitopes of an HIV protein. Polyepitope peptide chains comprising several CTL epitopes are particularly suitable.

In the context of the present invention, the expression “CTL epitope of an HIV protein” is intended to mean the sequences derived from the structural and regulatory proteins of HIV which induce a response mediated by the CD8+ cells of the immune system, such as the epitopes derived from the Env, Gag, Pol, Tat and Nef proteins. Any CTL epitope of HIV as defined above may be used in the context of the present invention. Such epitopes may be identified using the algorithms described for this purpose in the literature. By way of nonlimiting example of CTL epitopes which may be used in the lipopeptides according to the invention, mention may be made of the epitopes listed in table 4 of application WO 99/27954. The epitopes present in the lipopeptides according to the invention may be derived from a single strain of HIV or, preferably, from various strains of HIV, preferably from strains of primary isolates. When a single strain is used, these epitopes are derived from the conserved regions of the HIV genome.

In the context of the present invention, the expression “T-helper epitope of HIV” is intended to mean the sequences derived from the structural and regulatory proteins of HIV which induce a helper response mediated by the CD4 cells of the immune system. Any T-helper epitope which satisfies the definition above may be used in the context of the present invention.

According to a preferred embodiment, the lipopeptide according to the invention corresponds to a mixture in which the peptide chains are derived from the Env, Gag, Pol and Nef proteins of HIV, and in particular are derived from Gag, Pol and Nef.

The lipid chain is saturated or unsaturated and linear or branched, and comprises from 8 to 20 carbon atoms. It is preferably linear and saturated. The lipid chain preferably derives from a fatty acid of formula COOH—(CH₂)ₙ—CH₃, with n=12-16, in particular n=14, or from an alkyl halide of formula CH₂X—(CH₂)ₘ—CH₃ with X=Br or Cl and m=12-16, preferably with m=14. According to a particularly advantageous embodiment, the lipid chain is a palmitic acid.

The lipid chain is linked by covalent attachment at the C-terminal or at the N-terminal of the peptide chain directly or via one or more amino acid(s) preferably selected from the group consisting of lysine, lysine amide, cysteine, serine and threonine; preferably via a single amino acid corresponding to a lysine, a lysine amide or a cysteine.

When the lipid chain derives from a fatty acid, the COOH function of the fatty acid may be linked directly to the αNH₂ or εNH₂ function at the N-terminal of the peptide chain, by an amide attachment. Preferably, the lipid chain is linked via an amino acid, preferably a lysine, at the N-terminal or at the C-terminal of the peptide chain, by amide attachments. In the case of an attachment at the C-terminal of the peptide chain, said attachment is advantageously produced via a lysine-amide residue, which makes it possible to simplify the peptide synthesis process.

When the lipid chain derives from an alkyl halide as defined above, it may be linked to the peptide chain at the C-terminal or at the N-terminal, preferably via a cysteine, by a thioether attachment.

The lipopeptides according to the present invention may be synthesized by any conventional method. Methods which may be used in the context of the present invention are described in particular in documents U.S. Pat. No. 5,019,383, FR 90/15870, U.S. Pat. No. 5,993,823 and WO 99/27954 to which reference may be made for a complete description of the synthetic processes. Methods for synthesizing the lipopeptides according to the present invention are also described in the following references: “Yeast binding protein farnesyltransferase. Binding of S-alkyl peptides and related analogs”. Rozema et al in Organic Letters 1(5), 815-817 (1999); “Angiotensin analogues palmitoylated in position 1 and 4” Maletinska et al in J. Med. Chem. 40, 3271-3279 (1997); “Synthetic peptide vaccines: palmitoylation of peptide antigens by a thioesther bond increases imunogenicity” Beekman et al in J. Pept. Res. 50, 357-364 (1997); and “Synthesis and structural characterization of human-identical lung surfactant SP-C protein” Mayer-Fligge et al. in J. Peptide Sci 4, 355-363 (1998) to which reference may be made for a description of the techniques.

A subject of the present invention is therefore the administration of a composition comprising at least a lipopeptide as defined above and a pharmaceutically acceptable diluent or support.

The composition administered preferably comprises at least two different lipopeptides and in particular at least 5 different lipopeptides. The lipopeptides contained in such a mixture differ in their peptide chain, each lipopeptide comprising at least one CTL epitope which is different from the CTL epitope(s) present on the other lipopeptides of the mixture. The lipopeptides present in the mixture may also differ in their lipid chain. Preferably, all the lipopeptides consist of the same lipid chain. According to a preferred aspect of the invention, the lipid chain is a C16 chain. According to a particularly preferred embodiment, the lipid chain derives from a palmitic acid which is linked via a lysine or lysine amide residue to one of the ends of the peptide chain.

According to a preferred embodiment, the mixture according to the present invention consists of lipopeptides as defined in FIG. 1. The applicant has demonstrated, surprisingly, that the lipopeptides according to the invention are effective in the absence of any lipopeptide containing a universal T-helper epitope as described in WO 99/27954.

The lipopeptides are present in the mixture in equal weight amounts, preferably in equimolar amounts.

The mixture of lipopeptides is prepared from the individual lipopeptides in lyophilized form, in the following way; the lipopeptides are solubilized in pure acetic acid and mixed according to a defined order, i.e. starting with the least hydrophobic and ending with the most hydrophobic. An example of preparation of a mixture according to the invention is given in the examples which follow.

The term “pharmaceutically acceptable diluent or support” is used in its conventional sense and may, for example, represent, for an injectable solution, water, a buffered saline solution or a glucose solution. The pharmaceutically acceptable diluent or support will be selected as a function of the pharmaceutical form chosen, of the mode and route of administration and of pharmaceutical practice. The suitable supports or diluents and also the requirements in terms of pharmaceutical formulation are described in detail in Remington's Pharmaceutical Sciences, which represents a reference work in this field.

The lipopeptides according to the present invention can be administered via any conventional route normally used in the vaccines field, such as the parenteral (intradermal, intramuscular, subcutaneous, etc) route. The lipopeptides according to the invention are preferably administered via the intramuscular route. The administration may be carried out by injecting a single dose or repeat doses, for example and preferably 4 doses on D0 and at 1 month, 2 months and 3 months. Booster injections may optionally be administered, preferably every three months.

The lipopeptides according to the invention are administered in an amount of 50 micrograms to 3 milligrams of total lipopeptides, this being for the four injections and for the booster injections.

In the context of the present invention, the lipopeptides may advantageously be administered simultaneously or sequentially with a DNA vaccine or an attenuated recombinant virus vaccine.

In the case of the DNA vaccine, any plasmid vector containing eukaryotic virus regulatory elements may be used as a eukaryotic expression vector. It is possible to use any vector which allows the expression of proteins under the control of the SV40 “early” or “late” promoter, of the metallothionein promoter, of the human cytomegalovirus, murine mammary tumor virus or Rous sarcoma virus promoter, of the polyhedrin promoter or of other promoters which are effective for expression in a eukaryotic cell.

The therapeutic amounts of plasmid DNA can be produced by fermentation in E. coli, followed by purification. Aliquots from the working cell bank are used to inoculate the growth medium and are cultured until saturation in flasks with shaking or in a bioreactor according to well-known techniques. The plasmid DNA can be purified using a standard bioseparation method, such as a solid-phase anion exchange resin from QIAGEN, Inc. (Valencia, Calif.). If necessary, the supercoiled DNA can be isolated from the circular or linear open forms using an electrophoresis gel or any other method suitable for this purpose.

The purified plasmid DNA can be prepared for injections using varied formulation. The simplest is reconstituting the lyophilized DNA in sterile phosphate buffer (PBS). This method, named “naked DNA”, is preferably used for intramuscular (IM) administrations in the context of the present invention.

In the context of the present invention, the plasmid vector contains sequences encoding one or more peptides or proteins containing epitopes, at least some of which are common with those present in the lipopeptide(s) administered.

In order to maximize the immunotherapeutic effects of the DNA minigene vaccines, it may be desirable to employ another method for formulating the purified plasmid DNA. Cationic lipids in the formulation as described in WO 93/24640 may, for example, be used. In addition, glycolipids, fusogenic liposomes, peptides and compounds which are cited as protective overall, which are interactive and which do not condense, may also be mixed with the plasmid DNA so as to act on variables such as stability, intramuscular dispersion or the targeting of specific cells or organs.

According to a particularly advantageous embodiment, the lipopeptides according to the present invention are administered sequentially or simultaneously with an attenuated recombinant virus vaccine. Preferably, the lipopeptides and the attenuated recombinant virus vaccine are administered either simultaneously or sequentially according to a prime-boost method in which the attenuated recombinant virus is administered as primary immunization.

The expression “attenuated recombinant virus vaccine” is intended to mean a composition comprising an attenuated recombinant virus and a pharmaceutically acceptable diluent or support.

In the context of the present invention, an “attenuated recombinant virus” is a virus which has been genetically modified using modern techniques of molecular biology, for example restriction endonucleases and treatment with ligases, and which has thus been made less virulent than the wild-type virus by the deletion of certain genes or which has been attenuated by serial passages on a cell line derived from a host which is not the natural host or on primary permissive cells, or at low temperature.

The attenuated recombinant viruses according to the present invention express at least one CTL epitope of an HIV protein and at least one T-helper epitope preferably specific for HIV. The viruses preferably express the Gag, Env and protease proteins and CTL epitopes of Pol and Nef. Among the attenuated recombinant viruses which may be used in the context of the present invention, mention may be made, by way of nonlimiting example of adenoviruses, adeno-associated viruses, alphaviruses and poxviruses.

The attenuated virus acts as a vector for an immunogenic retroviral protein by virtue of the capacity of the virus to encode foreign DNA. The virus preferably induces a helper response and a cytotoxic response against the cells infected with HIV.

The virus is then introduced into the human body using conventional methods for immunizing with a live virus. A live virus vaccine may, for example, be administered at approximately 10⁴-10⁸ particles/dose, or from 10⁵ to 10⁹ pfu per dose. The real dose of such a vaccine may easily be determined using a conventional vaccinology assay.

In the context of the present invention, poxviruses are preferably used. A detailed review concerning these vectors is provided in U.S. Pat. No. 5,863,542, to which reference may be made for a complete description of the latter. A representative example of a recombinant poxvirus is ALVAC.

The DNA inserted into these vectors encodes the HIV antigens which comprise at least one of the following epitopes: HIV-1 Gag(+protease)(LAI), gp120(MN)(+the transmembrane component of gp41), Nef(BRU)CTL, Pol(LAI)CTL. ALVAC preferably comprises at least one CTL epitope of Nef and at least one CTL epitope of Pol (reverse transcriptase). The CTL epitopes of Nef and of Pol are preferably the Nef1, Nef2, Pol1, Pol2 and Pol3 CTL epitopes. In addition, sequences encoding Tat and/or Rev may advantageously be added. In the list above, the viral strains from which the antigens derive are in brackets.

The DNA sequences encoding the HIV antigens as defined above may derive from any known strain of HIV (HIV1 and HIV2, preferably HIV1), including the laboratory strains and primary isolates. The sequences of the CTL epitopes of Nef and of Pol identified above are described in U.S. Pat. No. 5,990,091 and correspond to the following sequences:

MPLTEEAELE LAENREILKE PVHGVYYDPS KDLIAEIQKQ
GQGQWTYQIY QEPFKNLKTG: Pol-3 CTL epitope (60 aa)
MEWRFDSRLA FHHVARELHP EYFKNC: Nef-2 CTL epitope
(26 aa)
MA IFQSSMTKIL EPFRKQNPDI VIYQYMDDLY VGSDLEIGQH
RTKIEELRQH LLPWGLTT: Pol-2 CTL epitope (60 aa)
MV GFPVTPQVPL RPMTYKAAVD LSHFLKEKGG LEGLIHSQRR
QDTLDLWIYH TQGYFPDWQN YTPGPGVRYP LTFGWCYKLV P:
Nef-1 CTL epitope (83 aa)
MIETVPVKL KPGMDGPKVK QWPLTEEKIK ALVEICTEME
KEGKISKIGP: Pol-1 CTL epitope (49 aa)

In the context of the present invention, ALVAC vCP 1433 or 1452 will preferably be used. The structure of these two ALVACs and also the process for manufacturing them are described in detail in U.S. Pat. No. 5,990,091, to which reference will be made for a complete description of these ALVACs.

According to another embodiment, an immunomodulator, preferably IL2 is administered sequentially or simultaneously. IL2 is preferably administered after the treatment with the lipopeptides, and preferably as 5 courses of 5 days, in a proportion of 4.5 million units twice a day.

The antiretroviral therapy is preferably stopped approximately 4 weeks after the final injection of lipopeptides. If the immunotherapy by lipopeptide administration is followed by IL2 treatment, preferably treatment comprising 5 courses, the antiretroviral therapy is stopped approximately 8 weeks after the final course, i.e. 28 weeks after the final injection of lipopeptides, in the case of a dose program comprising 4 injections (D0, 1 month, 2 months and 3 months).

The present invention will be described in greater detail in the examples which follow. The examples described below are given purely by way of illustration of the invention and can in no way be considered to limit the scope of the latter.

### EXAMPLE 1

Preparation of a Composition of Lipopeptides According to the Invention.

A mixture comprising 5 lipopeptides comprising peptide sequences derived from the Nef, Gag and Pol proteins of the LAI strain of the HIV virus, corresponding to the Nef 66-97, Nef 116-145, gag 17-35, gag 253-284 and pol 325-355 sequences is prepared according to the method described below.

For each lipopeptide, a lysine was added to the C-terminal end of the peptide and a palmitic acid was grafted onto the side chain of this lysine (amide attachment). The synthesis is carried out on solid phase (polystyrene-type resin comprising tricyclic amide substituents) using the Fmoc strategy. The amino acids are added from the C-terminal end to the N-terminal end. At the end of synthesis, the peptide is detached from the resin and the side chains are deprotected by treatment with trifluoroacetic acid.

The peptide is purified by HPLC using two different solvent systems. The pure peptide is then subjected to ion exchange chromatography so as to exchange the trifluoroacetate ions with acetate ions. The peptide is filtered (0.22 μm) and distributed into 100 mg bottles and lyophilized.

Each lipopeptide is then solubilized by adding pure acetic acid to obtain a concentration of 12.5 mg net/ml. The Nef 66, Nef 116 and Gag 17 lipopeptides are soluble in pure acetic acid. The Gag 253 and Pol 325 lipopeptides are not soluble in pure acetic acid. It is therefore necessary to introduce a sonication step in order to facilitate the solubilization of the latter two. If the aggregates are not dissociated, the sonication is continued, separating each period of sonication (30 s) by 30 s gaps of no sonication, during which the bottles are shaken manually. At the end of the sonication step, the suspensions should be homogeneous (but will not be clear). Each lipopeptide is then diluted by adding water for injectable preparation. If the solution obtained is not totally clear, steps of heating in a water bath (at 40° C.±2° C.) and sonication (1 min maximum) may be carried out, separating them by 30 s gaps of no sonication. The final preparation is obtained by mixing the lipopeptides in equal weight amounts, adding them in the following order (the most hydrophilic to the most hydrophobic lipopeptides):

Nef 66; Nef 116; Gag 17; Pol 325; Gag 253

The mixture is maintained with stirring (using a magnetic stirrer-bar) while the various lipopeptides are added. The mixture to be filtered should be clear. It is preferable to place it in a water bath at 40° C.±2° C. (5 minutes maximum) until a perfectly liquid and clear mixture is obtained. This must not exceed 10 min. The mixture is then filtered and distributed into bottles, in a proportion of 500 μg of each lipopeptide per bottle, and then lyophilized.

In order to prepare the composition according to the invention, the solution is reconstituted by adding 1 ml of a solution containing glucose at 5% (W/V), which may or may not be buffered.

The composition thus obtained comprises 2.5 mg of total lipopeptides per ml.

### EXAMPLE 2

Preparation of a Composition According to the Invention.

A composition comprising, besides the lipopeptides mentioned in example 1, a lipopeptide TT830-846, in which the peptide chain consists of the 830-846 epitope of the tetanus toxin, is prepared according to the method described in example 1. This epitope is described in the literature as being a universal T-helper epitope.

The lipopeptide TT830-846 consists of a C16 lipid chain derived from a palmitic acid, linked at the C-terminal of the peptide chain and synthesized according to the method of synthesis described above for the other lipopeptides. In the case of the lipopeptide TT830-846, the N-terminal end is acetylated, this being in order to avoid cyclization of the glutamine (Q) to pyroGlu.

The lipopeptide TT830-846 is added to the final preparation after the lipopeptide GAG17.

The composition thus obtained comprises 3.0 mg of total lipopeptides per ml.

### EXAMPLE 3

Immunotherapy of HIV+ Individuals

Primary-infected and chronically infected HIV+ individuals having a viral load of less than or equal to 1,000 copies per ml of plasma and a CD4+ level of greater than or equal to 300 cells per mm³ were subjected to a method of immunotherapy according to the present invention.

The following dose program was used:

Intramuscular injection on D0 and at 1 month, 2 months and 3 months, of 1 ml of the composition of example 1 or of 1 ml of the composition of example 2.

ALVAC-HIV (vCP1433) is co-administered intramuscularly at a dose of 10^{6.5} TCID50, this same dose being used for the 4 injections.

A portion of the individuals are subjected, after the immunotherapy, to IL2 treatment of 3 courses or 5 courses of 5 days, in a proportion of 4.5 million units twice a day.

The antiretroviral therapy is stopped 4 weeks after the final injection of lipopeptides or, when the latter is followed by IL2 treatment, for example of 5 courses, 8 weeks after the final course, i.e. 28 weeks after the final immunization.

The effectiveness of the immunotherapy, demonstrated by the at least temporary control, of the viremia, is determined by measuring the viral load according to the method described above.

The induction of an HIV-specific cytotoxic response is demonstrated using a method for measuring intracellular gamma INF (by FACS) or secreted gamma INF (ELISPOT).

The induction of a CD4-cell-mediated HIV-specific response is demonstrated using the method of lymphoproliferation in the presence of HIV antigen.

The proliferation of the CD4+ T lymphoctyes with respect to HIV is measured by culturing the cells in the presence of HIV antigens and measuring the incorporation of tritiated thymidine after culturing for 7 days.

The capacity of the blood mononucleated cells to proliferate is verified by culturing the cells with a mitogen or a control antigen (tetanus antigen for example).

In order to determine the subpopulation responsible for the proliferation, the CD4 cells are purified on magnetic beads and the proliferation assay is carried out on this subpopulation thus purified.

We claim:

1. A method for controlling viral rebound in HIV+individuals after termination of anti-retroviral therapy, the method comprising administering to an HIV+ individual who has undergone anti-retroviral therapy that has been terminated and which HIV+ individual has a viral load of less than or equal to 10,000 copies per ml of plasma and a CD4+ level greater than or equal to 300 cells per mm³, at least one lipopeptide comprising a peptide chain of 7 to 100 amino acids which comprises at least one CTL epitope of an HIV protein and which is linked by covalent attachment, optionally via a linker moiety, to a lipid chain comprising from 8 to 20 carbon atoms.

2. The method as claimed in claim 1, wherein the HIV+ individual has a viral load of less than or equal to 50 copies per ml of plasma and a CD4 level greater than or equal to 500 cells/mm³.

3. The method as claimed in claim 1, wherein the lipid chain comprises 16 carbon atoms.

4. The method as claimed in claim 1, wherein the lipid chain is linked to the peptide chain via a lysine or lysine amide residue linker moiety.

5. The method as claimed in claim 1, wherein a mixture of at least 5 different lipopeptides having CTL epitopes derived from the Nef, Gag and Pol proteins of HIV are administered.

6. The method as claimed in claim 5, wherein the lipopeptide mixture comprises the following lipopeptides:
(SEQ. ID. NO.: 1)
VGFPVTPQVPLRPMTYKAAVDLSHFLKEKGGLKΣ(Palm)-NH₂
(SEQ. ID. NO.: 2)
HTQGYFPDWQNYTPGPGVRYPLTFGWLYKLKΣ(Palm)-NH₂
(SEQ. ID. NO.: 3)
EKIRLRPGGKKKYKLKVIHKΣ(Palm)-NH₂
(SEQ. ID. NO.: 4)
NPPIPVGEIYKRWIILGLNKIVRMYSPTSILDKΣ(Palm)-NH₂, and
(SEQ. ID. NO.: 5).
AIFQSSMTKILEPFRKQNPDIVIYQYMDDLYKΣ(Palm)-NH₂.

7. The method as claimed in claim 5, wherein the mixture comprises at least one lipopeptide having a peptide chain comprising of a ubiquitous helper epitope.

8. The method as claimed in claim 1, wherein the lipopeptide or lipopeptides are administered intramuscularly at a dose of 50 μg to 3 mg of total lipopeptides on D0, and at 1 month, 2 months and 3 months.

9. The method according to claim 8, wherein each administration is in 4 doses totaling 50 μg to 3 mg.

10. The method as claimed in claim 1, wherein the lipopeptide or lipopeptides are co-administered with an attenuated recombinant virus vaccine.

11. The method as claimed in claim 10, wherein said attenuated recombinant virus is ALVAC.

12. The method as claimed in claim 11, wherein the ALVAC is vCP1433 or vCP1452.

13. The method as claimed in claim 1, wherein an immunomodulator is administered sequentially or simultaneously.

14. The method of claim 13, wherein the immunomodulator is IL2.
